(19) United States
(12) Patent Application Publication
(10) Pub. No.: US 2003/0098249 A1
(43) Pub. Date: May 29, 2003

(54) Brace case

(76) Inventor: Debra Anita Rollock, Miramar, FL (US)

Correspondence Address: DEBRA ANITA ROLLOCK, 2481 SW 161st AVENUE, MIRAMAR, FL, 33027, US

(21) Appl. No.: 10/307,179

(22) Filed: Nov. 29, 2002

Related U.S. Application Data
(60) Provisional application No. 60/333,852, filed on Nov. 28, 2001.

(51) Int. Cl. A61B 19/02

(52) U.S. Cl. 206/63.5; 206/361

(57) ABSTRACT

An oral hygiene system is provided which includes a decorative, compact, portable storage case comprising all the components of the oral hygiene system; it includes a hinged top with a mirror located on the interior surface of the hinged top, along with several brackets that will securely hold all items contained in the storage case. Items to be stored would be geared towards “braces-wearers” and would include (but not be limited to): a toothbrush, toothpaste, mouthwash, dental floss, dental floss threader, rubber bands, smile wax, bracket brush and toothpicks made to sufficient size to be contained and stored within the small portable case. This storage case also has an outer casing, made of the same material, with a clip on device attached to it in order to provide more convenient use by wearing clipped onto a belt, purse, pocket, or wherever one desires to place it.

This application claims benefit of my provisional application No. 60/333,852 filed on Nov. 28, 2001.

### BACKGROUND OF THE INVENTION

For years people have been concerned with the issue of oral hygiene while away from home, particularly individuals that wear braces; on many occasions people are in need of oral hygiene but they are not in a location where oral hygiene may be carried out comfortably or sanitarily for that matter. In many cases people often find themselves carrying several items to school, work, restaurants and other places in order to maintain oral hygiene throughout their daily routines, and those times when “impromptu” oral hygiene is necessary, as well. This is true of people with and without braces.

This has been a problem because so many of these items such as: mouthwash, toothbrushes, toothpaste, floss tend to be bulky and are usually carried separately, in several different carriers, or in one large bag. Furthermore, use of these products can be very inconvenient due to the places in which they are being utilized, usually in a public place: such as a public restroom (which is normally very unsanitary) or somewhere else, where things really can't be “set up” to be used comfortably.

Also, prior to the present invention there has not been a kit that contained all materials essential for people (particularly “braces-wearers) to “conveniently” carry all of the tools necessary for maintaining oral hygiene and comfort, in a manner that is both compact and convenient, while being fashionable and expressive of one's own style. For individuals that wear braces this is critical, due to the fact that individuals who wear braces are always subject to finding themselves in need of oral hygiene. Every time a person that is wearing braces eats a meal (no matter where they are) they will have food/substances trapped in their teeth or between their brackets and sometimes, depending what they are eating or drinking (such as spaghetti sauce or coffee) there is always a concern about “discoloration” of brackets (especially in the case of “clear” brackets—which are the most popular for both children and adults at the present time), as well as, teeth.

I have included relevant patents regarding this field of technology and I feel that the present invention differs from the prior art due to the fact that the “Brace Case” is designed especially for individuals that wear braces, which is very important because prior to the present invention there has never been a case comprising all of the necessary items needed for an individual with braces to conduct oral hygiene, as well as, the items necessary to relieve the major discomforts that “braces wearers” experience daily, securely held and stored in a very decorative, compact storage case with matching, detachable outer casing with clip on device for convenience and ease of use, as set forth herein. For example, there are kits that include dental floss, but for an individual with braces a “dental floss threader” is absolutely necessary, as is “smile wax”, which once applied, relieves the pain of loose wires piercing the tongue and inner mouth, covering brackets that have shifted due to the movement of the teeth causing discomfort, as well as, a “bracket brush” needed to brush between the brackets, an area that a regular toothbrush could not accommodate. And the case will have some form of ventilation whether it will be small holes/slits/lines in the top and/or bottom surfaces of decorative storage case itself can be used solely by being placed in a pocket or purse or the preferred embodiment is that it can be placed in the matching, detachable outer casing with clip on device, which would also have some form of ventilation, furthering its convenience by being clipped onto a belt or purse strap, making it totally “hands free” because you don't need to have a purse or pockets or a bag to carry it in or clip it on to for that matter, as well as, being worn as an accessory visible to the user and other persons.

The relevant patents regarding this area of technology are as follows:

Machacek et al U.S. Pat. No. 6,450,328 which discloses a dental equipment and material storage and multipurpose display apparatus.

Barth U.S. Pat. No. 5,924,562 which discloses an oral hygiene system.

Kelly et al U. S. Pat. No. 6,089,861 which discloses carrier for supporting orthodontic appliances.

Simister U. S. Pat. No. 6,484,732 which discloses carrying system for dental hygiene supplies.

Gustavel U.S. Pat. No. 5,348,028 which discloses compact multi-use oral hygiene device.

Wright U.S. Pat. No. 6,220,253 which discloses dental implement storage assembly.

As seen from the several patents there has never been an oral hygiene system as the present that presents a “specific” focus on the needs of individuals that wear braces, providing all of the essential components needed for braces wearers to conduct adequate oral hygiene maintenance and discomfort relief throughout their daily routines and at the same time creates efficiency and extreme ease of use when used with matching, detachable outer casing with clip on device, as well as, a very decorative, expressive and stylish accessory that can be worn on any and every occasion, being that this decorative storage case and matching outer casing with clip on device is being made with the knowledge that more adults than ever before are making an investment in their appearances by wearing braces in their latter years, as well as, teens and young children—which is why the fact that the present invention provides this decorative storage case in such a wide variety of colors, materials, prints and styles because it allows the user not only to express his or her own individual style, (obviously individual tastes will differ between adults, young children and teens) but it also allows the user to have a case for every occasion.

### BRIEF SUMMARY OF THE INVENTION

This case provides a solution to the problem of oral hygiene, away from home, being inconvenient in many ways with a specific focus on “braces wearers”.

First of all, the present invention is compact enough to be carried physically on a person's body; when utilized with detachable matching outer casing with clip on device can be clipped onto a belt, placed in a pocket, a purse, briefcase, or where ever user desires which makes this case virtually hands free because a purse, bag or pockets are not needed in order to transport with ease—it could even be clipped onto a bikini or swim trunks, which not only makes it ideal for daily maintenance, but it is also quite a “space saver” when traveling.

Secondly, the present invention contains all items one could possibly need to maintain oral hygiene whether they wear braces or not. Although the case does provide components essential for the individuals that wear braces, it also contains items that are beneficial for everyone.

Thirdly, the present invention is also very sanitary, being that all items are conveniently and securely held into place within the case, so they are not being carried around openly exposed to various germs, nor do they have to be placed on any unclean surfaces while in use. The case will also provide a means for ventilation by way of small holes/slits/lines in top, bottom or sides outer surface of decorative case and matching, detachable outer casing with clip on device to prevent any bacteria from building up due to wet bristles/dampness. The case also contains a mirror, so that the individual does not have to use a public restroom if he or she does not desire to do so.

And last, but not least, not only does this case provides convenience for the person who holds oral hygiene as a high priority, but it provides a sense of individual style for the person who is “fashion conscious”. Being that the case itself, as well as, the outer casing with clip on device is provided in a wide range of colors, materials, styles and prints. Both adults, children and teens will be able to wear this case as an accessory, accentuating his or her own personal style.

Overall, the present invention will provide a sense of higher self-esteem. Because it relieves individuals that wear braces of the continual fear of always having something trapped in their teeth or brackets or of having discoloration due to something they have eaten or had to drink (such as spaghetti sauce or coffee), it also provides anyone that uses it with a sense of cleanliness and a positive feeling that they have made a great investment in themselves and their appearance.

### WRITTEN DESCRIPTION

The “Brace Case” is a compact, decorative case used to “conveniently” transport and store materials essential to maintaining oral hygiene. The “Brace Case” is for anyone who considers oral hygiene a priority, but it is particularly geared towards individuals that wear braces, due to the lack of and high demand for, this type of product.

The purpose of this case is to provide a convenient, easily accessible means to transport all of the necessary items needed to maintain oral hygiene away from home (particularly for “braces wearers”) and at the same time making it very compact (by having all components made of substantial size) and very decorative, providing what can be viewed as an “accessory”, being made of various types of materials (for decorative purposes) giving the consumer (both children, adults and teens) the option of choosing a pattern or design that they feel reflects their own individual taste; creating a sense of expression, style and higher self-esteem.

The “Brace Case” can (but is not limited to) range in size from 3″-15″ wide, 2″-12″ from top to bottom and from 1″-10″ in diameter.

The case and matching detachable casing with clip on device would be (but not limited to) rectangular shape or circular, square, triangular or even oblong.

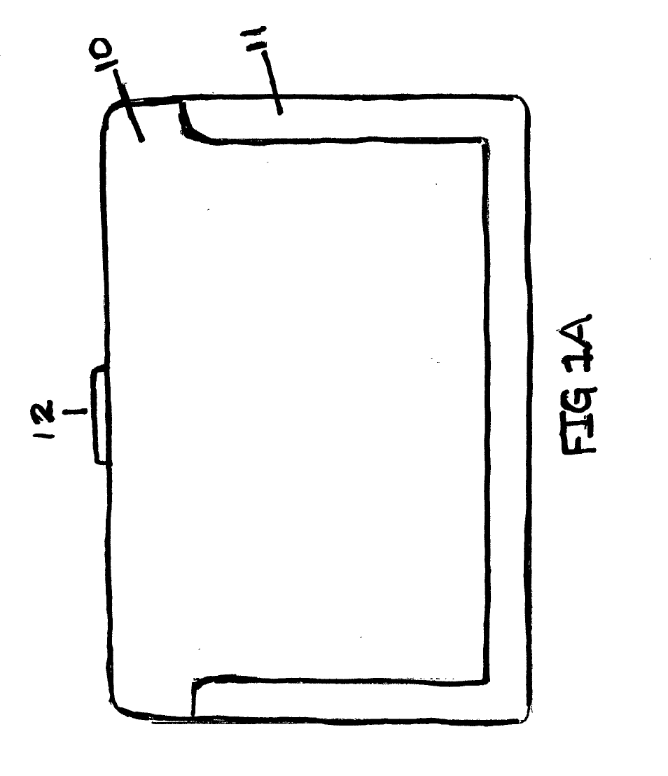

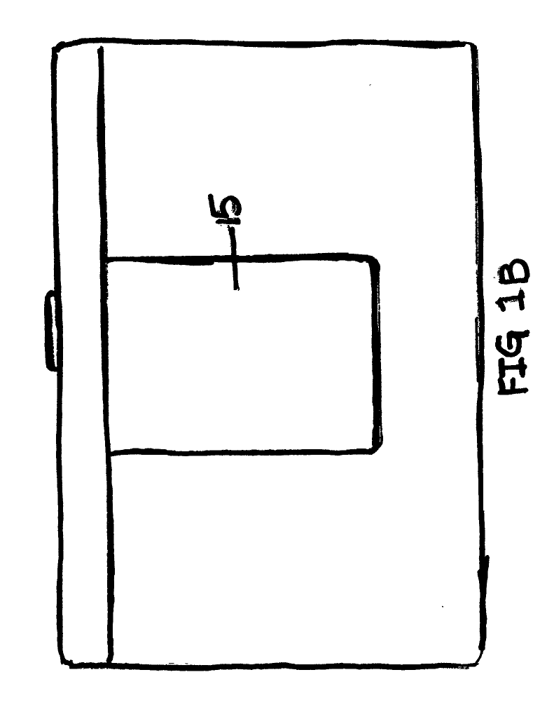

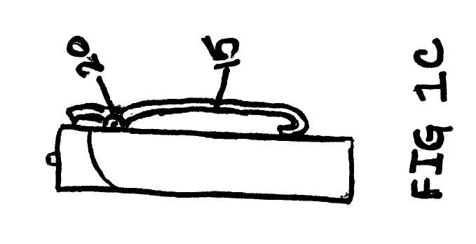

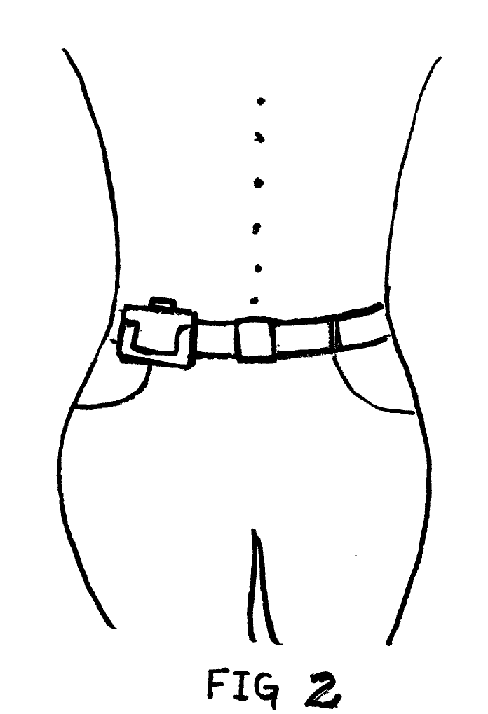

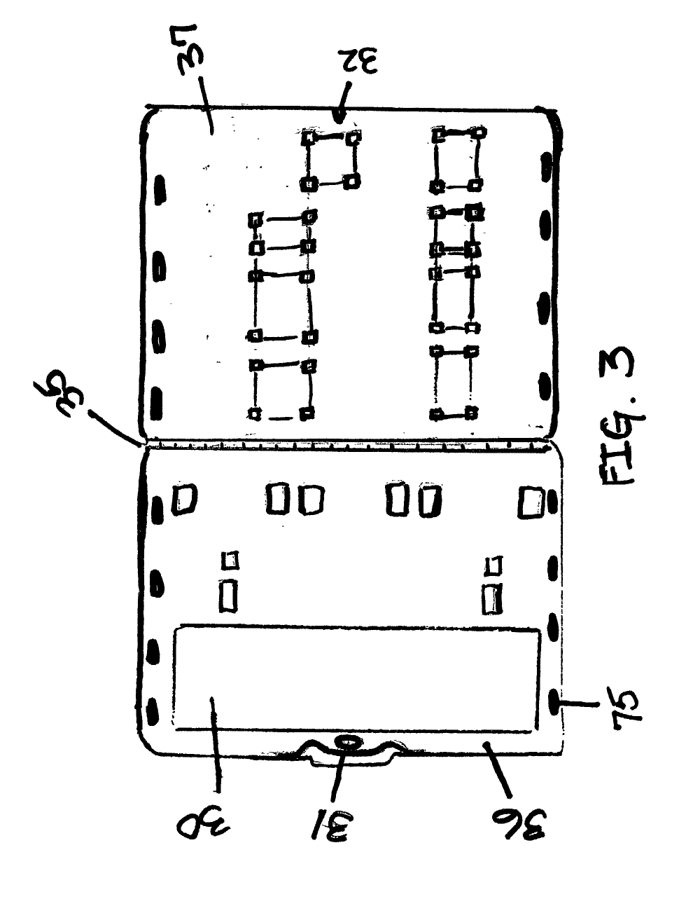

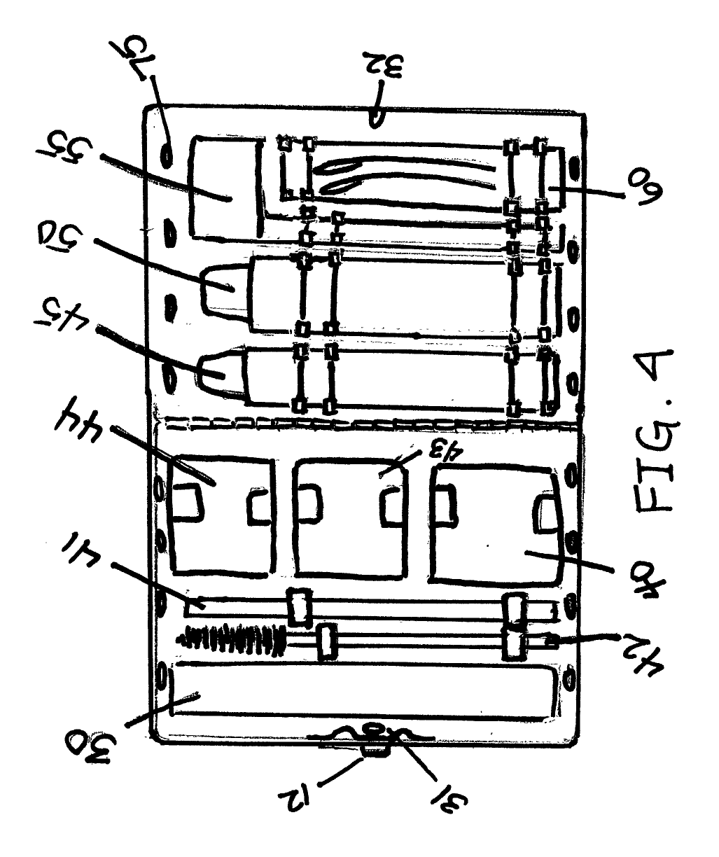

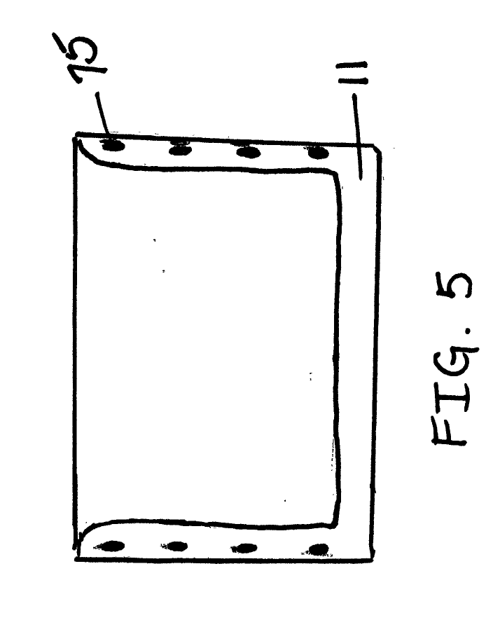

The case will have a decorative hinged top that will open and close preferably by way of clasp and button device (as shown in FIG. 5) or snap latch, spring, tie, velcro, buttoned, a magnetic device which will secure hinged lid when closed or even a handle by which it can be carried.

This case would contain all necessary components of an oral hygiene system that would accommodate the “braces-wearer” specifically or any other individual that believes maintaining oral hygiene is a priority. The components are made to comprise substantially the quantity appropriate for use and made to predetermined size to fit strategically and securely within decorative storage case and are as follows: a plastic toothbrush, a plastic container of mouthwash, a cylinder/spindle or dispenser containing dental floss, a plastic dental floss threader, a plastic bracket brush, a container of smile wax, a container/pouch comprising a supply of orthodontic rubber bands, individually wrapped or a pack/plastic tube of toothpicks, and a mirror mounted on/attached to the interior surface of the decorative storage case and possible means for ventilation, can be (but is not limited to) small holes/lines/slits in top, bottom or sides of the outer surfaces of the decorative storage case and the matching detachable outer casing with clip on device.

The case itself can be made out of metal, aluminum, steel, copper, brass, nickel, wood, plastic, leather, denim, animal skin, gemstone, fabric, and possibly adorned with gemstones, stones, feathers, studs or fur for decorative purposes.

The matching detachable outer casing with clip on device would be made of the same materials that the case it is displaying would be made of which could be metal, aluminum, steel, copper, brass, nickel, wood, plastic, leather, denim, animal skin, gemstone, fabric, and possibly adorned with gemstones, stones, feathers, studs or fur for decorative purposes.

The components of the oral hygiene system will be strategically, conveniently and securely held within the case; preferably held in by a plurality of latch type brackets/receptacles for holding (as shown in FIGS. 6 and 7) or can be tied, glued, velcroed, magnetized, held in pockets, placed in canisters/tubes; components within can possibly be made of the same colors and materials to match the decorative storage case and outer casing with clip on device (in color, print, material and texture).

Best/Preferred Embodiment is that this decorative hinged top storage case can be placed in matching detachable outer casing with clip on device. By wearing it this way it can be utilized in several different ways: clipped onto a belt, pocket, purse strap, back pack, bikini, swim trunks or wherever user so desires. And this way the “Brace Case” will be completely visible to the user and other persons being worn now as an accessory, in a fashionable manner, displaying a wonderful sense of style, as well as, being transported totally and completely “hands free” creating an extreme ease of use.

It is noted that the embodiment of the present invention, the “Brace Case”, an oral hygiene system as described herein in detail for exemplary purposes is of course subject to many different variations in structure, design, application and methodology. Because many varying and different embodiments may be made within the scope of the inventive concept(s) herein taught, and because many modifications may be made in the embodiment herein detailed in accordance with the descriptive requirements of the law, it is to be understood that the details herein are to be interpreted as illustrative and not in a limiting sense.

a) The portable system of claim 3, wherein matching outer casing has open frontal area so that when decorative storage case is slidably placed inside outer casing with clip on device, the decorative hinged top of storage case can be viewed by user and others, whereby being displayed as an accessory.

b) The portable system of claim 3, wherein matching outer casing is device for conveniently transporting decorative hinged top storage case in hands free manner.

c) The portable system of claim 3, wherein said is of predetermined size and shape for decorative hinged top storage case to fit slidably into outer casing with clip on device.

d) The portable system of claim 3, further comprising all necessary oral hygiene products of predetermined size includes a plastic toothbrush, a tube of toothpaste, a plastic container of mouthwash, a container of smile wax, a dental floss dispenser, a small pouch comprising a supply of rubber bands, a container of toothpicks, a plastic bracket brush, and a plastic dental floss threader, securely held within the interior of the storage case.

e) The portable system of claim 3, wherein said includes small holes on either side of outer surface on decorative storage case and matching outer casing whereby providing ventilation.

I claim:

1. A portable decorative storage case for oral hygiene products particularly for individuals that wear braces comprising all components necessary to maintain oral hygiene, which includes holding device that keeps all components held securely in place on interior surface of storage case.
a) The storage case of claim 1, wherein said comprising a plurality of holding devices of snap latch type for keeping all oral hygiene products held securely in place.
b) The storage case of claim 1, further comprising all necessary oral hygiene products of predetermined size includes a plastic toothbrush, a tube of toothpaste, a plastic container of mouthwash, a container of smile wax, a dental floss dispenser, a small pouch comprising a supply of rubber bands, a container of toothpicks, a plastic bracket brush, and a plastic dental floss threader, securely held within the interior of the storage case.
c) The storage case of claim 1, wherein said further comprising decorative hinged top which includes an interior surface with a mirror mounted on the interior surface.
d) The storage case of claim 1, wherein the decorative storage case is made of steel.
e) The storage case of claim 1, wherein said has a decorative outer surface comprising small holes for ventilation purposes.
f) The storage case of claim 1, wherein the decorative storage case is rectangular in shape.
g) The storage case of claim 1, further comprising clasp and button device for opening and closing purposes whereby pushing button down releases clasp and allows storage case to open.

2. An outer casing comprising a clip on device whereby allowing ease of use.
a) The outer casing of claim 2, further comprising clip on device attached to rear surface of outer casing by way of spring.
b) The outer casing of claim 2, wherein said is made of steel.
c) The outer casing of claim 2, wherein said is of rectangular shape.
d) The outer casing of claim 2, wherein said has open frontal area providing visibility.

3. A portable system comprising a decorative hinged top storage case including matching outer casing with clip on device for oral hygiene products, particularly for individuals who wear braces, comprising all components necessary to maintain oral hygiene with holding device within interior of decorative hinged top storage case to keep all components securely held in place.
